(19) United States
(12) Patent Application Publication
(10) Pub. No.: US 2009/0281294 A1
(43) Pub. Date: Nov. 12, 2009

(54) PROCESS FOR PRODUCTION OF COMPOUND LABELED WITH RADIOACTIVE FLUORINE

(75) Inventors: Keiichi Hirano, Chiba (JP); Daisaku Nakamura, Chiba (JP)

Correspondence Address: FITCH, EVEN, TABIN & FLANNERY, P. O. BOX 18415, WASHINGTON, DC, 20036, US

(73) Assignee: Nihon Medi-Physics Co., Ltd., Tokyo (JP)

(21) Appl. No.: 12/086,147

(22) PCT Filed: Nov. 30, 2006
(86) PCT No.: PCT/JP2006/323933

§ 371 Date: Jun. 6, 2008

(51) Int. Cl. C07H 5/02

(52) U.S. Cl. 536/18.4

(57) ABSTRACT

A process for the production of [¹⁸F]-TAFDG can produce [¹⁸F]-TAFDG in a high radiofluorination yield. A process for the production of an organic compound labeled with radioactive fluorine includes the steps of preparing a reaction solution containing [¹⁸F]fluoride ions, a phase transfer catalyst, potassium ions, 1,3,4,6-tetra-O-acetyl-2-O-trifluoromethanesulfonyl-β-D-mannopyranose as a labeling precursor compound and a solvent, and giving a reaction condition to the reaction solution to obtain 1,3,4,6-tetra-O-acetyl-2-[¹⁸F]fluoro-2-deoxyglucose, in which the solvent contains water.

### TECHNICAL FIELD

The present invention relates a process for production of organic compounds labeled with radioactive fluorine, specifically 1,3,4,6-tetra-O-acetyl-2-[¹⁸F]fluoro-2-deoxyglucose and its deprotected product, 2-[¹⁸F]fluoro-2-deoxy-D-glucose.

### BACKGROUND ART

Nuclear medicine examination represented by positron emission tomography (hereinafter referred to as PET) and single photon emission computed tomography (hereinafter referred to as SPECT) is effective in diagnosing a variety of diseases including cancers. These examination techniques involve administrating an agent labeled with a specific radioisotope (hereinafter referred to as radiopharmaceutical) to a patient followed by detecting γ-ray emitted directly or indirectly from the agent. Nuclear medicine examination is characteristic in that it has not only high specificity and sensitivity to diseases, but also an advantage of providing information on the function of lesions, compared to other examination techniques.

For example, 2-[¹⁸F]fluoro-2-deoxy-D-glucose (hereinafter referred to as ¹⁸F-FDG), one of radiopharmaceuticals used for PET examination, tends to be concentrated in areas where glucose metabolism is enhanced, thereby making it possible to specifically detect tumors in which glucose metabolism is enhanced.

Among the above-mentioned nuclear medicine examinations, PET provides images of higher quality and thus can provide images higher in diagnosis performance, compared to SPECT that has been widely used conventionally in clinical use. PET examination is, therefore, expected as a new diagnostic modality succeeding SPECT examination, and development of radiopharmaceuticals for PET examination (hereinafter referred to as PET diagnostic agent) is now carried out in many research facilities and the like. For example, various receptor mapping agents and blood flow diagnostic agents have been synthesized and are under investigation for clinical application.

The PET diagnostic agent is an agent that contains, as an effective component, a compound labeled with a positron emitting nuclide such as ¹¹C, ¹⁵O and ¹⁸F. The most widely used compound among them is an organic compound labeled with ¹⁸F represented by ¹⁸F-FDG. There have been reported various methods for production of the representative compound, ¹⁸F-FDG, and most of them are generally classified into a method proposed by Hamacher (hereinafter referred to as Hamacher method) and an on-column method.

The Hamacher method is a method wherein [¹⁸F] are activated by evaporating a solution containing [¹⁸F], potassium carbonate and a phase transfer catalyst to dryness, then a solution in acetonitrile of 1,3,4,6-tetra-β-acetyl-2-O-trifluoromethanesulfonyl-β-D-mannopyranose (hereinafter referred to as TATM) that is a labeling precursor is added and heated to give an intermediate, 1,3,4,6-tetra-O-acetyl-2-fluoro-2-deoxyglucose (hereinafter referred to as ¹⁸F-TAFDG), followed by subjecting the intermediate to a deprotection process and a purification process to give targeted ¹⁸F-FDG. On the other hand, the on-column method is a method wherein ¹⁸F-FDG is obtained by performing activation of [¹⁸F] and ¹⁸F labeling reaction in a column followed by deprotection and purification (see Patent Document 1, Non-Patent Document 1 and Non-Patent Document 2).

It is disclosed that when ¹⁸F-FDG is synthesized by the above mentioned method, complete dehydration is required in the step in which the solution containing ¹⁸F, potassium carbonate and a phase transfer catalyst is evaporated to dryness to activate ¹⁸F (see Non-Patent Document 1 and Patent Document 2).

In addition, it is disclosed that if dehydration is insufficient in the above mentioned step of activating ¹⁸F in the synthesis of an organic compound labeled with ¹⁸F, ¹⁸F may be hydrated, thereby reducing nucleophilicity of ¹⁸F and causing decrease of yield (see Patent Document 3).

Patent Document 1: Japanese Patent Laid-Open (Kokai) No. 6-157572.

Patent Document 2: Japanese Patent Laid-Open (Kohyo) No. 11-508923.

Patent Document 3: Japanese Patent Laid-Open (Kokai) No. 5-345731.

Non-Patent Document 1: Hamacher K., Coenen H. H., Stocklin G., “Efficient Stereospecific Synthesis of No-carrier-added-2-[¹⁸F]fluoro-2-deoxy-D-glucose Using Aminopolyether Supported Nucleophilic Substitution,” J. Nucl. Med., 1986, 27, 2, p. 235-238.

Non-Patent Document 2: K. Hamacher et al., “Computer-aided Synthesis (CAS) of No-carrier-added-2-[¹⁸F]Fluoro-2-deoxy-D-glucose: an Efficient Automated System for the Aminopolyether-supported Nucleophilic Fluorination” Applied Radiation and Isotopes, (Great Britain), Pergamon Press, 1990, 41, 1, p. 49-55.

### DISCLOSURE OF THE INVENTION

### Problem to be Solved by the Invention

Among the representative methods for producing ¹⁸F-FDG, the Hamacher method has a feature such that it can achieve a relatively high yield, but nevertheless has a problem such that production yield may greatly vary in some cases. The primary cause of this variation is the variation of yield of ¹⁸F labeling reaction, that is, the variation of production yield of ¹⁸F-TAFDG (hereinafter referred to as radiofluorination yield). Therefore, in order to stably supply ¹⁸F-FDG commercially, it is necessary to adopt a method or condition that enables stable production in high yield, and it is necessary for that purpose to establish a condition in which ¹⁸F-TAFDG can be stably produced in high yield.

The present invention has been made in light of the above situation, and has aimed at providing a process for production of ¹⁸F-TAFDG, which can achieve a high radiofluorination yield.

### Means for Solving the Problem

As a result of diligent studies, the inventors have completed the invention by finding that the above mentioned problem can be solved and a high radiofluorination yield can be achieved by allowing a reaction solution for fluorination reaction to contain a certain amount of water.

According to the conventional knowledge, the presence of water in the reaction solution acts as a factor that inhibits ¹⁸F fluorination reaction, and therefore it has been suggested that complete dehydration is required in the ¹⁸F activation step (that is, a step of obtaining a mixture containing [¹⁸F]fluoride ions, a phase transfer catalyst and potassium ions) which is performed prior to ¹⁸F fluorination reaction in the synthesis of ¹⁸F-FDG (see Japanese Patent Laid-Open (Kohyo) No. 11-508923 and Japanese Patent Laid-Open (Kokai) No. 5-345731). On the contrary to such a conventional knowledge, we have found that the yield in the ¹⁸F fluorination reaction can be improved and stabilized by allowing a certain amount of water to co-exist in the reaction solution and thus completed the present invention.

A process for production of an organic compound labeled with radioactive fluorine according to the present invention is a process comprising the steps of: preparing a reaction solution containing [¹⁸F]fluoride ions, a phase transfer catalyst, potassium ions, TATM as a labeling precursor compound, and a solvent; and giving a reaction condition to the above-mentioned reaction solution to obtain ¹⁸F-TAFDG, characterized in that the above-mentioned solvent contains water.

As the above-mentioned solvent of the reaction solution, an amphipathic organic solvent can be used which is capable of dissolving TATM and contains a certain amount of water; typically a liquid mixture of acetonitrile and water can be used.

Amount of water to be contained in the solvent is preferably 500 μg/g to 10000 μg/g, more preferably 750 g/g to 9500 μg/g, still more preferably 1000 μg/g to 9000 μg/g, particularly preferably 1000 μg/g to 3000 μg/g. The case where the amount of water contained in the solvent is too low is not preferable, because fluorination reaction does not proceed sufficiently. The case where the amount is too much is not preferable either, because decrease in fluorination reaction yield and increase in impurity are brought about.

In the production process according to the present invention, the step of preparing a reaction solution is performed typically by preparing a mixture containing [¹⁸F]fluoride ions, a phase transfer catalyst and potassium ions beforehand, and adding TATM and a solvent to the mixture respectively. It is preferable that TATM is previously dissolved in a solvent, and then added. The step of preparing a reaction solution is not, however, limited to these methods. Water to be contained in the solvent is preferably added simultaneously with or prior to the addition of TATM. Specifically, the step of preparing a reaction solution can be performed by adding TATM as a labeling precursor compound to a system in which water or a water-containing solvent is added to the mixture of [¹⁸F]fluoride ions, a phase transfer catalyst and potassium ions. The introduction of water to the above described system before the addition of TATM to the system enables a more stable and higher ¹⁸F fluorination yield.

### EFFECT OF THE INVENTION

It has become possible to obtain ¹⁸F-TAFDG in a high radiofluorination yield by use of the process for production of an organic compound labeled with radioactive fluorine according to the present invention.

### BEST MODE FOR CARRYING OUT THE INVENTION

Hereinafter, the most preferable embodiment will be described about the process for production of an organic compound labeled with radioactive fluorine according to the present invention.

In the process for production of an organic compound labeled with radioactive fluorine according to the present invention, a mixture containing a phase transfer catalyst, [¹⁸F]fluoride ions and potassium ions is obtained in the first place. [¹⁸F]fluoride ions can be obtained by a known method, for example, a method in which H₂¹⁸O enriched water is used as a target and exposed to a proton bombardment. In this instance, [¹⁸F]fluoride ions exist in the H₂¹⁸O enriched water used as a target. This H₂¹⁸O enriched water containing [¹⁸F]fluoride ions is passed through an anion-exchange column to collect radioactive fluorine by adsorption to the column, and thus separated from the H₂¹⁸O enriched water. Then, a potassium carbonate solution is run through the column to elute the [¹⁸F]fluoride ions, and supplemented with a phase transfer catalyst and dried to solid to obtain a mixture containing a phase transfer catalyst, [¹⁸F]fluoride ions and potassium ions.

Amount of potassium carbonate to be used here may be about 0.3 or more in terms of molar ratio relative to the labeling precursor TATM, but should not be excessive because it causes decrease of yield unfavorably. In the most preferable aspect, concentration and amount of the potassium carbonate solution are adjusted so that the molar ratio of potassium ion to TATM is 0.3 to 4.

Various compounds having a property to form a clathrate with ¹⁸F ion may be used as a phase transfer catalyst. Specifically, various compounds used for production of organic compounds labeled with radioactive fluorine may be used; 18-crown-6-ether and other various aminopolyethers may be used. In the most preferable aspect, CRYPTOFIX 222 (trade name, manufactured by Merck Ltd.) may be used.

After a mixture containing a phase transfer catalyst, [¹⁸F]fluoride ions and potassium ions is obtained, a reaction solution containing the mixture and the labeling precursor compound TATM, is prepared. The process according to the present invention is characterized in that the solvent in the reaction solution prepared in this step comprises water. In the most preferable aspect, this step is performed by adding water-containing acetonitrile to the above-described mixture followed by addition of TATM.

Amount of water to be contained in acetonitrile is adjusted so that concentration of water is 500 to 10000 μg/g in the prepared reaction solution. Amount of the solvent to be added is not specifically restricted as long as it is sufficient to dissolve the above-mentioned mixture, however, it must be noted that if the amount is too much, too much processing time is required for the heat and evaporation step for preparing ¹⁸F-FDG from ¹⁸F-TAFDG.

The method for adding TATM is not specially limited, but a method in which TATM is first dissolved in acetonitrile and then added is preferable from the viewpoint of operability. In this case, it is preferable that acetonitrile which is used to dissolve TATM contains water in an amount as small as possible, from the viewpoint of inhibiting decomposition of TATM.

After the completion of preparation of the reaction solution, labeling reaction is performed by giving a reaction condition to the reaction solution. The reaction condition may be a condition which is sufficient for progressing ¹⁸F fluorination reaction of TATM by means of heating or the like. In the production of ¹⁸F-TAFDG, labeling with ¹⁸F can be usually achieved by heating a vessel containing a reaction solution at 85° C. for about 5 minutes in a hermetic state. ¹⁸F-TAFDG can be synthesized by this procedure.

When ¹⁸F-FDG is prepared by deprotection of ¹⁸F-TAFDG, a step may be performed in which the reaction vessel is left open after the completion of labeling reaction, and the reaction solution is heated to evaporate the solvent followed by addition of an acid and heating. In addition, the obtained ¹⁸F-FDG may be subjected to a purification process if required.

### EXAMPLES

Hereinafter, the present invention will be described in further detail with reference to Examples; however, it should be understood that the details of the Examples are not intended to limit the present invention.

### Examples 1-11

A target water enriched with ¹⁸O was subjected to proton bombardment to obtain [¹⁸F]fluoride ions in a form of a target water containing [¹⁸F]fluoride ions. This target water containing [¹⁸F]fluoride ions was measured for the amount of radioactivity using CRC-15R (trade name, manufactured by CAPINTEC, Inc.) (denoted as the amount of radioactivity A). Then, after this target water was passed through an anion-exchange column to collect [¹⁸F]fluoride ions by adsorption, a potassium carbonate solution was passed through the column to elute [¹⁸F]fluoride ions. This eluate containing [¹⁸F]fluoride ions was supplemented with a solution of CRYPTOFIX 222 (trade name, manufactured by Merck Ltd.) in acetonitrile, heated and evaporated to dryness, to obtain a mixture containing [¹⁸F]fluoride ions, potassium ions and a phase transfer catalyst (CRYPTOFIX 222, trade name, manufactured by Merck Ltd.).

By adding 0.5 mL of acetonitrile mixed with water to the mixture, the mixture was dissolved therein so that the water content in the reaction solution was that shown in Table 1. The resultant solution was supplemented with 0.5 mL of a solution of TATM in acetonitrile (concentration: 40 mg/mL) to prepare the reaction solution. Then, the vessel containing the reaction solution was sealed and heated at 85° C. for 5 minutes using a heating block to perform the labeling reaction. After the completion of the reaction, the vessel was opened and heated further at 85° C. for 5 minutes to evaporate the solvent, and measurement of radioactivity was performed using CRC-15R (trade name, manufactured by CAPINTEC, Inc.) (referred to as the amount of radioactivity B).

The obtained reaction product was dissolved in acetonitrile with a water content of 1% (w/w) and analyzed by TLC under the following condition. The area percentage of ¹⁸F-TAFDG was considered as the radiochemical purity.

### TLC Analysis Condition:

TLC plate: Silica Gel 60 F₂₅₄ (trade name, manufactured by Merck Ltd.)
Mobile phase: chloroform/ethyl acetate=80/20
Detector: Rita Star (trade name, manufactured by raytest Co. Ltd.)

Using the obtained radiochemical purity and the amounts of radioactivity A and radioactivity B, the [¹⁸F]fluorination yield was determined according to the following equation (1). Here, the amounts of radioactivity A and radioactivity B corrected for decay in consideration of measurement time were used in the calculation.

$$[ {}^{18} F ] fluorination yield ( \% ) = \frac{B}{A} \times [ \begin{matrix}radiochemical purity \\ of {}^{18} F - TAFDG\end{matrix} ] \qquad ( 1 )$$

The results are shown in Table 1 and FIG. 1. As shown in the table and the FIGURE, the value of fluorination yield of ¹⁸F-TAFDG was as good as not less than 70% when the water content in the reaction solution was between 500 to 10000 μg/g. On the other hand, it was shown that when the water content in the reaction solution is less than 500 μg/g and when more than 9500 μg/g, the fluorination yield and the ¹⁸F-TAFDG value tended to decline.

From the results described above, it was shown that ¹⁸F-TAFDG can be produced in a good fluorination yield when the water content in a reaction solution is from 500 to 10000 μg/g. Especially, the fluorination yield of not less than 85% and the radiochemical purity of not less than 90% were achieved when the water content was between 750 and 9500 μg/g.

TABLE 1

| | Water content | Radiochemical | Fluorination |
|---|---|---|---|
| | in the reaction | purity of ¹⁸F- | yield of ¹⁸F- |
| | solution μg/g | TAFDG % | TAFDG % |
| Example 1 | 500 | 85.69 | 82.64 |
| Example 2 | 750 | 94.01 | 90.46 |
| Example 3 | 1000 | 95.33 | 92.23 |
| Example 4 | 1500 | 95.80 | 93.67 |
| Example 5 | 2000 | 97.29 | 94.56 |
| Example 6 | 2500 | 95.30 | 91.18 |
| Example 7 | 3000 | 96.27 | 89.24 |
| Example 8 | 5500 | 95.80 | 87.69 |
| Example 9 | 8500 | 93.91 | 86.41 |
| Example 10 | 9500 | 94.95 | 88.62 |
| Example 11 | 10000 | 85.50 | 71.18 |

### INDUSTRIAL APPLICABILITY

The process for production of an organic compound labeled with radioactive fluorine according to the present invention can be employed in producing a radiopharmaceutical ¹⁸F-FDG, and is useful in the field of nuclear medicine examination.

### BRIEF DESCRIPTION OF THE DRAWING

FIG. 1 shows the relationship of the water content in the reaction solution with the radiochemical purity of ¹⁸F-TAFDG and the fluorination yield.

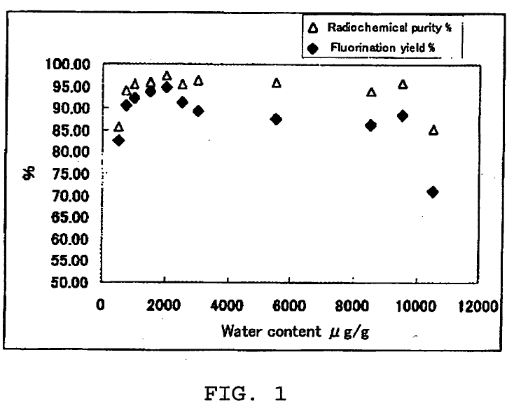

1. A process for production of an organic compound labeled with radioactive fluorine, which comprises the steps of:
preparing a reaction solution containing [¹⁸F]fluoride ions, a phase transfer catalyst, potassium ions, 1,3,4,6-tetra-O-acetyl-2-O-trifluoromethanesulfonyl-β-D-mannopyranose as a labeling precursor compound, and a solvent; and
giving a reaction condition to said reaction solution to obtain 1,3,4,6-tetra-β-acetyl-2-[¹⁸F]fluoro-2-deoxyglucose, in which said solvent contains water.

2. The process for production of an organic compound labeled with radioactive fluorine according to claim 1, in which said solvent is a liquid mixture of water and acetonitrile.

3. The process for production of an organic compound labeled with radioactive fluorine according to claim 1, in which the water content in said reaction solution is from 500 to 10,000 μg/g.

4. The process for the production of a compound labeled with radioactive fluorine, according to claim 1, in which said step of preparing a reaction solution comprises adding 1,3,4,6-tetra-O-acetyl-2-O-trifluoromethanesulfonyl-β-D-mannopyranose to a system in which water or a water-containing solvent is added to a mixture of [¹⁸F]fluoride ions, a phase transfer catalyst, and potassium ions.

5. The process for production of an organic compound labeled with radioactive fluorine according to claim 2, in which the water content in said reaction solution is from 500 to 10,000 μg/g.

6. The process for the production of a compound labeled with radioactive fluorine, according to claim 2, in which said step of preparing a reaction solution comprises adding 1,3,4,6-tetra-O-acetyl-2-O-trifluoromethane-sulfonyl-β-D-mannopyranose to a system in which water or a water-containing solvent is added to a mixture of [¹⁸F]fluoride ions, a phase transfer catalyst, and potassium ions.

7. The process for the production of a compound labeled with radioactive fluorine, according to claim 3, in which said step of preparing a reaction solution comprises adding 1,3,4,6-tetra-O-acetyl-2-O-trifluoromethane-sulfonyl-β-D-mannopyranose to a system in which water or a water-containing solvent is added to a mixture of [¹⁸F]fluoride ions, a phase transfer catalyst, and potassium ions.
